(19) United States
(12) Patent Application Publication
(10) Pub. No.: US 2005/0244427 A1
(43) Pub. Date: Nov. 3, 2005

(54) Isolation and production of bioactive marine products

(76) Inventor: Russell G. Kerr, Boca Raton, FL (US)

Correspondence Address: AKERMAN SENTERFITT, P.O. BOX 3188, WEST PALM BEACH, FL, 33402-3188, US

(21) Appl. No.: 11/064,018

(22) Filed: Feb. 18, 2005

Related U.S. Application Data
(60) Provisional application No. 60/546,481, filed on Feb. 20, 2004.

(51) Int. Cl. A61K 35/80; C12N 1/12

(52) U.S. Cl. 424/195.17; 435/257.1

(57) ABSTRACT

Biologically active compounds can be isolated from algal cells isolated from gorgonians. The algal cells can be cultured in vitro under conditions that promote production of high concentrations of biologically active compounds, including pseudopterosins.

### CROSS-REFERENCE TO RELATED APPLICATIONS

This application claims the benefit of Provisional Patent Application No. 60/546,481 entitled “ISOLATION AND PRODUCTION OF BIOACTIVE MARINE PRODUCTS” filed on Feb. 20, 2004, and is incorporated by reference herein its entirety.

### FIELD OF THE INVENTION

The invention generally relates to the fields of biochemistry, molecular biology, marine biology, and medicine. More particularly, the invention relates to the isolation and production of biologically active compounds from dinoflagellate symbionts of gorgonians.

### BACKGROUND

For decades, gorgonians (O. Gorgonacea, Ph. Cnidaria), corals commonly known as sea feathers, sea whips and sea fans, have been recognized as a rich source of chemically diverse compounds. Many of these compounds have been isolated by natural products chemists and are known to be useful for treating various diseases. Often of terpenoid origin, these compounds have displayed anti-inflammatory, anti-cancer, and anti-microbial properties. Because organic methods for synthesizing such molecules are far from being commercially practical, conventional methods of obtaining these compounds in purified form involve harvesting the coral, and then extracting and purifying the desired compound by conventional chemical methods. Unfortunately, the destruction of coral for this purpose has an undesirable impact on the environment.

### SUMMARY

The present invention relates to the development of a method and system for isolating and producing the biologically active compounds found in gorgonians without massive destruction of coral reefs. The invention is based on the discovery that certain of the dinoflagellate symbionts which live in symbiosis with gorgonians contain far greater concentrations of the biologically active compounds than does the coral itself. As described below, methods of culturing such symbiotic algae in vitro that allow the algae to be replicated to large numbers have been developed. When cultured under the appropriate conditions, these alga make the biologically active compounds at a level comparable to or higher than that found in naturally occurring coral. Using plant growth factors, production of the biologically active compounds can be significantly increased. The invention thus allows for the production of biologically active compounds from a renewable resource. Using the method and system of the invention should significantly reduce the cost of making such biologically active compounds, and should also reduce the destruction of coral reefs.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### BRIEF DESCRIPTION OF THE DRAWINGS

FIG. 1 is a graph displaying the amount of pseudopterosins produced by the algal cells at 4 time points during a 42 day culture.

FIG. 2 is a 2.5% agarose gel showing the restriction fragment length polymorphism (RFLP) analysis of initially isolated algal cells as well as that of algal cells that had been cultured in vitro for 8 weeks.

FIG. 3 is a 1.2% agarose gel displaying the ITS region analysis of initially isolated algal cells as well as that of algal cells that had been cultured in vitro for 26 weeks. Lane 1—1 kB ladder, Lane 3—negative control, Lane 6—initial alga isolate, Lane 9—cultured alga 26 weeks after inoculation, Lane 12—cultured alga 8 weeks after inoculation.

FIG. 4 is a graph showing the induction of fuscol production in Symbiodinium cells isolated from Eunicea fusca with Methyl Jasmonate, Giberellic Acid and Salicyclic Acid.

### DETAILED DESCRIPTION

The invention encompasses a method and system for isolating and producing the biologically active compounds found in gorgonians without massive destruction of coral reefs. In the invention, biologically active compounds are purified from algal cells isolated from a host gorgonian rather than from the whole coral. Because the concentration of the biologically active compounds is higher in the algal cell fraction than in whole coral, methods for obtaining compounds in high purity are facilitated. Moreover, methods for culturing the algal cells isolated from the host gorgonian in vitro have been developed that preserve the cells ability to make biologically active compounds. Such culture methods allow large quantities of the algal cells to be produced as a source of biologically active compounds, such as bioactive terpenes.

Regarding the Culture of algae as a source of bioactive terpenes according to the invention, the observation that a culture of the dinoflagellate symbiont (Symbiodinium sp.) isolated from Pseudopterogorgia elisabetha produces pseudopterosins is of great significance as this represents the first such demonstration of natural product production. Little is known about the factors that control the production of natural products in the complex environment within invertebrate hosts. Interactions between invertebrate host, dinoflagellate, and bacteria (and possibly fungi) may be responsible directly or indirectly for the production of defensive chemicals. With the exception of data obtained by the Inventors there have not been any disclosed cases of natural product biosynthesis from any microorganism isolated from an invertebrate that the inventors are aware. Thus, the discovery by the Inventors of conditions for the culture of Symbiodinium isolated from P. elisabethae that maintains the ability of the algae to produce pseudopterosins is of great novelty and significance.

The below described preferred embodiments illustrate adaptations of these systems and methods. Nonetheless, from the description of these embodiments, other aspects of the invention can be made and/or practiced based on the description provided below.

### Algal Cells

Algal cells useful in the invention are available from the natural environment. For example, in the marine environment, symbiotic alga often form an association with a variety of marine organisms. Thus, alga may be obtained by harvesting from marine organisms such as Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Pocillopora, Rhodactis, Stylophora, Tridacna, and Zoanthus. Marine organism hosts can be of the class Octocorallia that can include West Indian octocorals of the genera Briareidae, Plexauridae, Gorgoniidae, Briarium, Erythropodium, Eunicea, Leptogorgia, Muricea, Phyllogorgia, Plexaura, Plexaurella, Pseudoplexaura, Pseudopterogorgia, Pterogorgia. Examples of specific algae for use in the invention include those isolatable from Pseudopterogorgia elisabethae, Euniceafusca, and Pseudopterogorgia bipinnata.

Alga useful in the invention include those that produce a pseudopterosin, seco-pseudopterosin, or pseudopterosin or seco-pseudopterosin biosynthetic intermediate. For example, the alga can be a dinoflagellate such as an alga of the genus Symbiodinium. Examples of Symbiodinium species include S. kawagutii, S. goreaui, S. muscatinei, S. pulchrorum, S. bermudense, S. californium, S. microadriatiucum, S. pilosum, S. meandrinae, S. corculorum, and S. linucheae. S. microadricum is preferred for use in the invention because, as shown herein, it is capable of producing pseudopterosins at high levels in in vitro culture. As not all clades of S. microadricum make high levels of pseudopterosins under the culture conditions described below, a preferred clade of S. microadricum is clade B.

Alga useful in the invention also include those that produce fuscol, fuscosides or biosynthetic intermediates leading to one or both of these metabolites. As with the previous example, the alga can be a dinoflagellate such as an alga of the genus Symbiodinium (same examples as listed for above case). S. microadriaticum is preferred for use in the invention because, as shown herein, it is capable of producing fuscol/fuscosides at high levels in in vitro culture. As not all clades of S. microadriaticum make high levels of fuscol/fuscoside under the culture conditions described below, preferred clade of S. microadricum is clade B.

Additional alga useful in the invention include those that produce kallolides, bipinnatins or biosynthetic intermediates leading to one or both of these groups of diterpenes. As with the previous example, the alga can be a dinoflagellate such as an alga of the genus Symbiodinium (same examples as listed for above case). S. microadriaticum is preferred for use in the invention because, as shown herein, it is capable of producing kallolides/bipinnatins at high levels in in vitro culture. As not all clades of S. microadriaticum make high levels of kallolides/bipinnatins under the culture conditions described below, preferred clade of S. microadricum is clade B.

### Isolation of Alga from Coral

Algal cells can be isolated from a marine organism by several conventional methods. For example, as described below alga cells are isolated from a host by grinding or blending the host in an aqueous medium such as filtered seawater, buffer, or culture medium. After the grinding or blending step, the algal cells are separated from the non-algal cells and debris by a suitable method, e.g., filtration and/or a density gradient separation. For example, a crude homogenate of coral can first be filtered through cheesecloth to remove cellular debris, and then subjected to repeated centrifugation (relatively dense algal cells collect in the pellet). Other purification steps that might be used include any method that separates cells based on cell size, density, surface charge or hydrophobic surface properties. Methods that maximally preserve cell viability are preferred. Specific examples of such methods include density gradient separation of cells using centrifugal elutriation [such as on silica particles coated with polyvinylpyrrolidone (Percoll®) or a non-ionic synthetic polymer of sucrose (Ficoll®)], partitioning between aqueous two-phase systems, flow cytometry, antibody-based methods (including magnetic, column, and panning techniques), and free flow electrophoresis according to known procedures in the art such as those described e.g., in Cell Separation: A Practical Approach (Practical Approach Series, 193), eds. Fisher, Francis, and Rickwood, Oxford University Press, New York, 1998.

For the alga described below, the following general method may be used to purify a dinoflagellate symbiont (Symbiodinium sp.) from a gorgonian. Either live gorgonian tissue or flash frozen material is used. If the latter is used, the frozen gorgonian is thawed in an ice water bath. The gorgonian is then homogenized in a blender in chilled deionized (DI) water and the resulting homogenate is strained into a centrifuge tube through cheese cloth. The filtrate is then centrifuged at about 250 g, the supernatant discarded and this procedure repeated about 5 times. The final pellet is highly enriched in Symbiodinium cells. This pellet is centrifuged over 100% Percoll and the visible cells collected using a plastic pipet and rinsed in DI water. The cells are then suspended in 10% Percoll and centrifuged again. The supernatant is discarded and the cells resuspended in DI water and rinsed. The latter Percoll centrifugation and rinsing steps are repeated until the supernatant is clear. The pellet recovered from this process is subjected to a discontinuous Percoll density gradient centrifugation (100%, 70%, and 30%) and the cells collected by pipet. Cells are readily apparent as a brown layer in the Percoll, typically, at the 30/70 interface. Purified cells can be examined for purify by light microscopy and standard staining methods used to examine viability.

### Culture of Algal Cells

Following the isolation of algal cells, the cells are rinsed with filtered seawater or another suitable aqueous solution to remove residue from the previous purification step. The viability of the cells can be examined utilizing any stain (e.g., Trypan Blue) or fluorescent dye which is ingested by the cell and gives a measure of the quantity of living cells present in the sample (see previous reference). The amount of viable cells is determined by observing a portion of the sample under a microscope and counting the number of viable cells manually or automatically using an instrument such as a flow cytometer. The cell viability can also be evaluated using cell viability/proliferation assays known in the art. The cells in each sample are aliquoted into cell culture media based upon the number of surviving cells and grown in culture under culture conditions.

Various media and growth conditions can be utilized for culturing algal cells. See, e.g., Rowan et al., Proc. Natl. Aca. Sci. USA. 89:3639-43, 1992, and references cited therein. Examples of culture medium that can support algal cell cultures include ASP-8A (Provasoli et al., Arch. Microbiol. 25: 392-428, 1957), Guillard's F/2 (Sigma), and Prov 50 (Guillard. Culture of phytoplankton for feeding marine invertebrates, In Smith and Chanley, (eds.) Culture of Marine Invertebrate Animals, Plenum Press, New York, 1975, pp 26-60; Guillard et al., Can. J. Microbiol. 8: 229-239, 1962; Provasoli, et al. Arch. Microbiol. 25: 392-428, 1957). Media can be supplemented with various nutrients, metals, and other additives (e.g., antibiotics) known in the art or described in the references herein. Culture conditions important for algal cell growth include light intensity, illumination cycle, and temperature. For example, conditions for the culture of the alga of the invention include a 14-10 hour light-dark cycle, a light intensity of 30-150 μmol photons m⁻² s⁻¹ and a temperature between 23-29° C.

Plant growth factors can be added to the cell cultures to increase the yield of biologically active compounds. Growth factors such as methyl jasmonate, giberellic acid, or salicylic acid can be added to algal cell cultures for a suitable period of time (e.g., 48 hours) to enhance the production of biologically active compounds.

### Purification and Quantification of Biologically Active Compounds

Biologically active compounds can be purified from algal cells isolated from a gorgonian after in vitro culture or after isolation from the coral. In an exemplary method of the invention, purified dinoflagellate cells are dried (lyophilization preferred) and then extracted using a suitable solvent (e.g., MeOH/CH₂Cl₂ 1:1; or other solvent of comparable polarity). The extract is then concentrated and purified by flash chromatography and/or HPLC (or other standard chromatographic methods). Standard spectroscopic methods, and comparison with literature data can be used to confirm the identity of purified compounds.

The biosynthetic capability of isolated dinoflagellates can be assessed by enzymology. In one example, purified frozen algal cells are thawed and incubated with ³H-labelled substrate (e.g., GGPP). Following an appropriate incubation period (1-24 hours), the reaction mixture is frozen with liquid nitrogen and placed on a lyophilizer to dry. The dried cells can be extracted with a variety of organic solvents and the biologically active compound (e.g., diterpene) is purified as described above. The radioactivity can then be measured using a scintillation counter and the radiochemical purity established by derivatization, further HPLC purification and scintillation counting.

The biologically active compounds present in the cells can be quantified by conventional methods, e.g., using high-performance liquid chromatography (HPLC). For example, pseudopterosins and seco-pseudopterosins as well as intermediates involved in the biosynthesis of these classes of compounds can be purified from the algal cells according to known procedures such as those described by Look et al., Proc. Natl. Acad. Sci. USA. 83:6238-6240, 1986; Look et al., J. Org. Chem. 51:5140-5145, 1986; Look et al., Tetrahedron 43:3363-3370, 1987; Roussis et al., J. Org. Chem. 55:4916-4922, 1990; and U.S. Pat. Nos. 4,849,410, 4,745,104, and 5,624,911.

Fuscol and fuscosides, as well as their biosynthetic precursors can be purified from the algal cells according to procedures developed for the gorgonian Eunicea fusca e.g. Gopichand et al., Tet. Letters 39:3641-3644, 1978; Shin et al., J. Org. Chem. 56:3153-3158, 1991. Kallolides and bipinnatins, as well as their biosynthetic precursors can be purified from the algal cells according to procedures developed for the gorgonians Pseudopterogorgia kallos and P. bipinnata e.g. Look et al., J. Org. Chem. 50:5741-5746, 1985; Rodriguez et al., J. Nat. Prod. 62:1228-1237, 1999.

In addition to these purification methods, other chromatographic techniques such as ion-exchange, size-exclusion, thin-layer, supercritical fluid, capillary electrophoresis, and gas chromatography could be used in the separation and analysis of biologically active compounds from the algal cells.

### EXAMPLES

The present invention is further illustrated by the following specific examples. The examples are provided for illustration only and are not to be construed as limiting the scope or content of the invention in any way.

### Example 1

### Isolation of the Algal Cells

A coral sample, Pseudopterogorgia elisabethae, was obtained from Sweetings Cay, Bahamas at a depth of about 10-15 meters. Live coral was homogenized (in seawater filtered with a 0.22 μm filter) with either a homogenizer or a blender. The crude homogenate was filtered through cheesecloth to remove large skeletal parts. The homogenate was then centrifuged at 1,000×g for 10 min. Following centrifugation, the supernatant was decanted and the algal cell pellet was resuspended in filtered seawater. This centrifugation process repeated at least 10 times.

The algal pellet was further purified using a discontinuous density gradient of colloidal silica coated with polyvinylpyrrolidone (Percoll®, Sigma). The Percoll® was diluted with filtered seawater and the gradient involved layering 10 mL of 30% on top of 10 mL of 70% Percoll®, on top of 5 mL of 100% Percoll®. The cell suspension (10 mL) was layered on top of the 30% layer and separated by centrifuging at 1000×g for 10 min. The algal cells trapped between the Percoll® layers were removed with a pipette. To remove any Percoll® residue, the purified alga was rinsed two additional times with filtered seawater.

### Example 2

### Cell Viability Tests and Cell Culture

Purified algal cells were stained with Trypan Blue (Sigma) solution to assess the cell viability. Using a hemocytometer, the cells were observed under a microscope and counted. If the cells were brown in color, they were considered to be alive and if they were blue or clear in color, they were considered to be dead. The concentration of the cells that were deemed to be viable was adjusted and the cells (˜10⁵-10⁶) were aliquoted into ASP-8A media (Provasoli et al., Arch. Microbiol. 25: 392-428, 1957). Antibiotic mix was then added to 1% of the final concentration of the solution. Antibiotic mix consisted of preparing a stock solution of the following ingredients (solubilized in ASP-8A media unless otherwise noted): polymixin (0.9 mg/mL, solubilized in EtOH), streptomycin (33 mg/mL), penicillin-G (5.8 mg/mL), neomycin (20 mg/mL), tetracycline (2.5 mg/mL), chloramphenicol (10 mg/mL, solubilized in 95% EtOH). These ingredients were then added in the following amounts and diluted to 100 mL with ASP-8A media: polymixin=1 mL, streptomycin=10 mL, penicillin-G=2.24 mL, neomycin=1 mL, tetracycline=0.1 mL, and chloramphenicol=5 μL. The cultures were maintained between 23-29° C. on a 14-10 light-dark cycle and illuminated with wide spectrum bulbs at 30-150 μmol photons m⁻²s⁻¹(Ei).

### Example 3

### Quantification of Pseudopterosins

Cells in culture (2×10⁵−3×10⁷) were dried on a lyophilizer, extracted with ethyl acetate, and the resulting dried crude extract partitioned between methanol/water (9:1). The methanol/water fraction was again partitioned between methanol/water (1:1) and methylene chloride. The resulting methylene chloride extract was analyzed using normal phase HPLC with a gradient elution (hexanes:ethyl acetate) of increasing ethyl acetate. The pseudopterosins (A-D) were identified by comparing the HPLC elution profile to a standard sample. Quantities of pseudopterosins (in mg) were determined from a calibration curve made using known amounts of pseudopterosins (A-D).

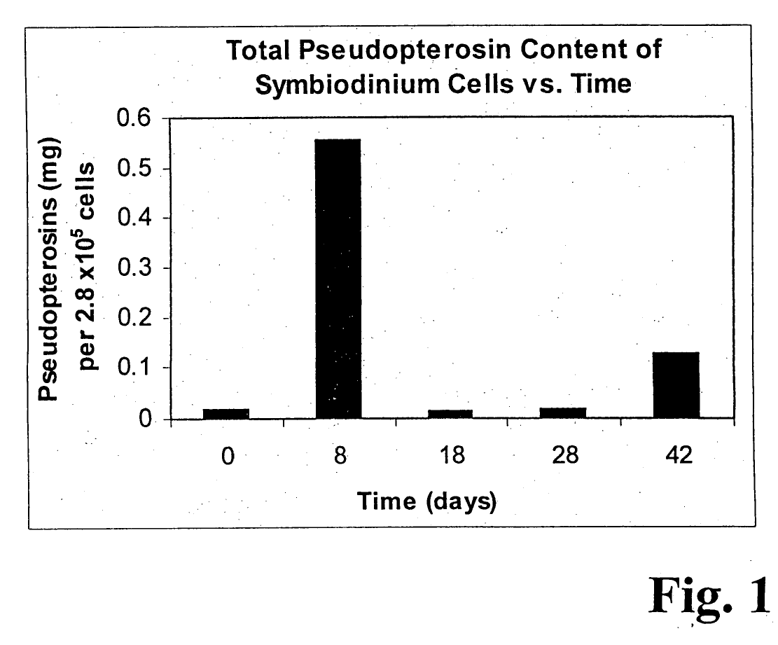

FIG. 1 is a graph displaying the amount of pseudopterosins present in the cells compared to the total amount of time the cells were in culture. The initial increase in the level of pseudopterosins per 2.8×10⁵ cells was typical due to the stress the cells endured from inoculation of the culture. Following the preliminary increase, the amount of pseudopterosins in the cells returned to a level comparable to the initial isolate. At day 42, the level of pseudopterosins present in the algal cells was more than 700% greater than that of the initial isolate indicating that the observed increase in pseudopterosin content was due to the production of pseudopterosins by the algal cells.

### Example 4

### PCR Amplification with Taq Polymerase and RFLP

Analysis of Initial Isolate and Algal Cell Culture The initial isolate of flash frozen P. elisabethae which corresponded to the same sample in culture was homogenized with liquid nitrogen using a mortar and pestle. The DNA was isolated using a DNA Plant Mini Kit for Isolation of DNA (Qiagen, Valencia, Calif.). PCR was performed using 10 μM SS5 and 5.6 μM SS3Z zooxanthallae-specific primers. The 10 μM SS5 primer used was 5′ggttgatcctgccagtagtcatatgcttg-3′ and the 5.6 μM SS3Z primer used was 5′-agcactgcgtcagtccgaataaatcaccgg-3′. The PCR conditions utilized were: 92° C. for 3 min followed by 30 cycles at 80° C. for 5 min, 92° C. for 30 s, 52° C. for 40 s, 72° C. for 30 s, and then a final cycle at 72° C. for 5 min. The PCR products were separated on a 0.8% agarose gel and stained with ethidium bromide. The DNA annealed between 1.65 and 2.0 kB and the specific product corresponded to 1.65 kB. The process was repeated for algal cells (which had been in culture for 8 weeks) and both products were excised from the gel and stored at −20° C.

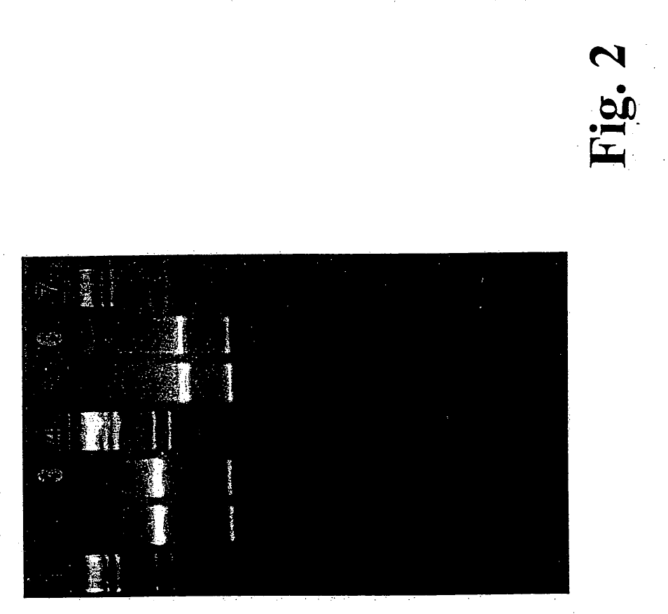

The PCR products from both the initial isolate and the cultured cells were digested with Taq 1 polymerase as well as a DPN II restriction enzyme. The Taq 1 polymerase digestion was conducted at 65° C. for 270 min and the DPN II digestion was performed at 37° C. for 240 min. The samples were separated on a 2.5% agarose gel and stained with ethidium bromide. FIG. 2 displays the agarose gel: Lane 1—1 kB ladder, Lane 2—Taq 1 digest of cultured alga 8 weeks after inoculation, Lane 4—1 kB ladder, Lane 5—DPN II digest of the initial algal isolate, Lane 6—DPN II digest of cultured alga 8 weeks after inoculation, Lane 7—1 kB ladder. The RFLP analysis results demonstrated that cells in culture 8 weeks after inoculation were still of the same clade (clade B) as the initial algal isolate (Toller et al., Biol. Bull. 201: 348-359, 2001; Santos, Taylor and Coffroth 2001 J. Phycol. 37, 900-912). Additionally, the results verified that the culture was homogenous and had not been overtaken by other microorganisms.

### Example 5

### ITS Region Sequence Analysis Analysis of Initial Isolate and Algal Cell Culture

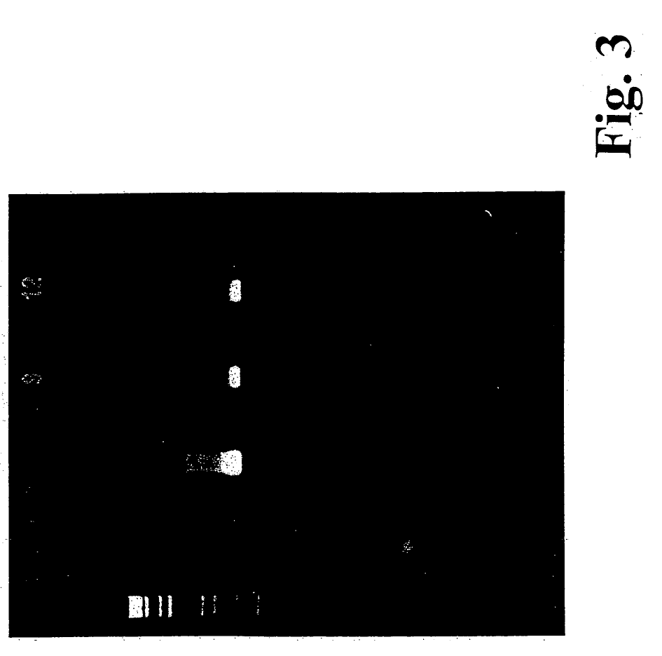

The DNA from the initial isolate as well as the cultured cells was isolated as described in Example 4. Primers specific for the ITS region (ITS ss5 and ITS ss3) were utilized for PCR. The ITS ss5 primer used was 5′-gcatcgatgaagaacgcagc-3′ and the ITS ss3 primer used was 5′gctgcgttcttcagcgat-3′. The PCR conditions were: 94° C. for 2 min followed by 30 cycles at 94° C. for 0.4 min, 53° C. for 1 min, and 72° C. for 1 min. The 30 cycles were followed by a final cycle at 72° C. for 30 min. The PCR products were analyzed using a 1.2% agarose gel and stained with ethidium bromide (FIG. 3). The results indicate that the phylogeny of the cells in culture 26 weeks after inoculation was congruent with the phylogeny of the initial algal isolate.

### Example 6

### Kallolide and Bipinnatin Content in Dry Gorgonian (Pseudopterogorgia bipinnata) vs. Dry Algae Cells

Algal cells from P. bipinnata were discovered to be a source of the diterpenes kallolides and bipinnatins. Algal cells isolated from P. bipinnata were shown to have a much higher concentration of the diterpenes kallolides and bipinnatins than is present in the crude coral tissue.

### Methods

300 mL of flash frozen P. bipinnata type A were removed from an ultrafreezer and thawed in an ice water bath. The tissue was homogenized in a Waring blender using ice cold DI water. The homogenate was then strained into 50 mL Falcon tubes and centrifuged at 250×g for 10 minutes. The supernatant was discarded and the pellet was resuspended in DI water and centrifuged again. The rinsing and centrifugation steps were repeated 5 times until the supernatant became clear. The cell pellet was then layered over 100% Percoll and centrifuged at 50×g for 15 minutes. Cells were collected using a plastic pipet, rinsed in DI water, suspended in 10% Percoll, and centrifuged at 200×g for 5 minutes. The supernatant was discarded and the cells were resuspended in DI water and rinsed. The Percoll and rinse steps were repeated until supernatant was clear. The cells were then layered on top of a discontinuous Percoll gradient (100%, 70%, and 30%) and centrifuged at 50×g for 20 minutes. Cells at the 30/70 interface were collected by pipet and rinsed in DI water and resuspended.

The cells were examined by light microscopy to assess cell purity then pelleted by centrifugation. The cells were resuspended in phosphate buffer and frozen at −80° C. The cells were thawed and incubated with 1 μCi ³H-GGPP for 20 hrs at 29° C. and 200 RPM. The cells were frozen with liquid nitrogen and placed on a lyophilizer to dry. Dry cells were extracted with MeOH:H₂O at a ratio of 9:1 and partitioned with Hexanes. The hexanes layer was dried and weighed. This layer weighed 17.55 mg. Water was added to make the MeOH:H₂O ratio 1:1 and partitioned with Methylene chloride. The CH₂Cl₂ layer was dried and weighed. This layer weighed 69.93 mg. The CH₂Cl₂ layer was passed through a silica pipet using a gradient from 100% hexanes to 50% EtOAc in 10% increments of 20 mL each. Kallolide A was collected using NP HPLC (100% Hex to 50:50 Hex:EtOAc over 30 min., hold 10 min). HPLC conditions were as follows: 2 mL/min, 270 nm, Vydac semi-prep column. The amount of Kallolide A collected was 6.34 mg, equal to 0.01931 mmol which is equal to 2062 DPM (equaling 1.07×10⁵ DPM/mmol). Kallolide A was derivatized as per the method described in Look et al., (Journal of Organic Chemistry, 50:5741-46, 1985) by catalytic hydrogenation/hydrogenolysis. The derivative was collected using RP HPLC (3% H₂O in MeOH, Beckman C18 semi-prep column, 2 mL/min, 270 nm). The weight of the derivative was 1.77 mg, equal to 0.00556 mmol, which is equal to 661 DPM (1.19×1 05 DPM/mmol). The reaction yield obtained was 29%.

### Results

From gorgonian tissue, 41.71444 g of dry bipinnata type A, 28.63 mg (0.0686%) of 2-Omethyl bipinnatin J, 27.20 mg (0.0652%) of Kallolide A, and 111.41 mg (0.267%) of Kallolide A acetate were obtained. From algal cells, 381.16 mg of dry zoox from P. bipinnata type A, 2.0 mg (0.525%) of 2-Omethyl bipinnatin J, 6.458 mg (1.694%) of Kallolide A, and 15.042 mg (3.946%) and Kallolide A acetate were obtained. These results demonstrate that the algal cells have the biosynthetic machinery to produce the kallolides and bipinnatins. Culturing algal cells isolated from P. bipinnata according to the method described above for P. elisabethae resulting in the production of kallolides.

### Example 7

### Summary of Fuscol Content in Dry Gorgonian (Pseudopterogorgia bipinnata) vs. Dry Algae Cells

Live Eunicea fusca was collected from Florida Keys. The tissue was homogenized in a blender using 50:50 DI/Filtered Seawater (FSW). The homogenate was strained into 50 mL Falcon tubes and centrifuged at 160×g for 5 minutes. The supernatant was discarded and the pellet was resuspended in 50:50 DI/FSW. The sample was centrifuged again, followed by three rounds of rinsing and centrifuging until the supernatant was clear. The cell pellet was layered over a Percoll gradient (30% and 70%) and centrifuged at 38×g for 10 minutes. Cells on the 30/70% interface were collected using a plastic pipet and rinsed two times in 50:50 DI/FSW water. Cells were examined by light microscopy to assess cell purity and to count the number of cells. 2.38×10⁸ cells (500 μL) were removed from the sample and diluted to 2 mL with tris buffer (20 mM containing 3 mM EDTA, 5 mM MgCl₂ and 5 mM β-mercaptoethanol). The sample was incubated with 1μ Ci ³H-GGPP for 24 hrs at 29° C. and 180 RPM. The cells were centrifuged and the buffer was removed using a syringe and filtered into a scintillation vial. The cells were extracted with methylene chloride and ethyl acetate (8 times) followed by filtration into the scintillation vial containing the buffer. The crude extract was dried and weighed. The dried extract weighed 7.08 mg. The extract was partitioned between MeOH:H₂O 9:1 and hexanes. The hexanes layer was dried and weighed. This layer weighed 4.77 mg. Water was added to make MeOH:H₂O at a ratio of 1:1 and partitioned with methylene chloride. The CH₂Cl₂ layer was dried and weighed. This layer weighed 1.67 mg.

Fuscol was collected using reversed phase HPLC with a phenyl hexyl semi-prep column. The conditions were as follows: 70:30 MeOH:H₂O, hold 5 min., to 90:10 MeOH:H₂O over 15 min., hold 10 min. (a total run time of 30 min.), 3 mL/min, 240 nm. The specific activity was 1.65×10⁶ DPM/mmol. The fuscol peak was reinjected, fractions were collected, and the radioactivity was counted using a scintillation counter. The collected fuscol weighed 420 μg, equal to 0.0014 mmol, which is equal to 3420 DPM (2.34×10⁶ DPM/mmol). These results demonstrate that the algal cells have the biosynthetic machinery to produce fuscol and fuscosides.

The fuscol content in dry Eunicea fusca tissue was found to be 0.57% (39.5 mg fuscol from 6.963 g dry gorgonian tissue). The fuscol content in dry algae cells was determined to be 242.2 mg dry zoox from Eunicea fusca and 7.54 mg (3.11%) fuscol. These results demonstrate that the algal cells have a much higher concentration of fuscol than is present in the crude coral tissue and provide evidence that these algae are the biosynthetic source of these terpenes.

### Example 8

### Plant Growth Factors Increase the Yield of Biologically Active Compounds in In Vitro Cultures of Algal Cells Isolated from Coral

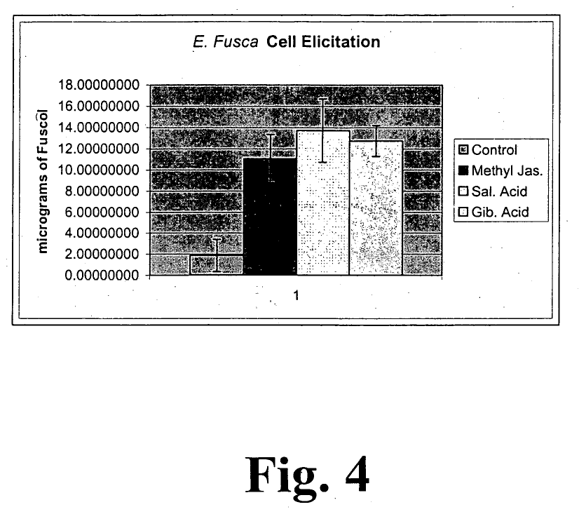

Referring to FIG. 4, the effect of growth factors on the production of fuscol in in vitro cultures of alga cells isolated from Eunicea fusca (as described above) was analyzed. Cells were isolated and maintained for 24 hours in culture medium as described above. One hundred μM methyl jasmonate, giberellic acid, or salicyclic acid was then added to the cells (control cells received no treatment) for 48 hours. The cells were then extracted and fuscol concentrations were determined by standard chromatographic methods (RP HPLC, detection at λ 240 nm). Significantly more fuscol was produced in cultures contacted with methyl jasmonate, giberellic acid, or salicyclic acid.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of isolating a biologically active compound, the method comprising the steps of:
culturing algal cells isolatable from a marine organism under conditions that promote replication of the cells and production of the biologically active compound; and
purifying the biologically active compound from the cultured algal cells.

2. The method of claim 1, wherein the alga is of the genus Symbiodinium.

3. The method of claim 2, wherein the species of Symbiodinium is selected from the group consisting of S. kawagutii, S. goreaui, S. muscatinei, S. pulchrorum, S. bermudense, S. californium, S. microadriatiucum, S. pilosum, S. meandrinae, S. corculorum, and S. linucheae. S. microadricum.

4. The method of claim 1, wherein the alga is obtained from a host marine organism.

5. The method of claim 4, wherein the host is selected from the group consisting of: Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Pocillopora, Rhodactis, Stylophora, Tridacna, and Zoanthus.

6. The method of claim 5, wherein the host is selected from the group consisting of: Pseudopterogorgia elisabethae, Eunicea fusca, and Pseudopterogorgia bipinnata.

7. The method of claim 2, wherein the Symbiodinium is of clade B.

8. The method of claim 1, wherein the step of culturing algal cells isolatable from a gorgonian under conditions that promote replication of the cells and production of the biologically active compound comprises culturing the cells in ASP-8A culture medium.

9. The method of claim 1, wherein the step of culturing algal cells isolatable from a gorgonian under conditions that promote replication of the cells and production of the biologically active compound comprises subjecting the cells to illumination using a 14-10 light-dark cycle.

10. The method of claim 9, wherein the light intensity of the illumination is 30-150 μmol photons m⁻²s⁻¹.

11. The method of claim 1, wherein the step of culturing algal cells isolatable from a gorgonian under conditions that promote replication of the cells and production of the biologically active compound comprises culturing the cells at a temperature between 23-29° C.

12. The method of claim 1, wherein the step of culturing algal cells isolatable from a gorgonian under conditions that promote replication of the cells and production of the biologically active compound comprises contacting the cells with at least one plant growth factor.

13. The method of claim 12, wherein the at least one plant growth factor is selected from the group consisting of methyl jasmonate, giberellic acid, and salicyclic acid.

14. A culture of algal cells isolatable from a marine organism.

15. The culture of claim 14, wherein the algal cells are of the genus Symbiodinium.

16. The culture of claim 15, wherein the species of Symbiodinium is selected from the group consisting of S. kawagutii, S. goreaui, S. muscatinei, S. pulchrorum, S. bermudense, S. californium, S. microadriatiucum, S. pilosum, S. meandrinae, S. corculorum, and S. linucheae. S. microadricum.

17. The culture of claim 14, wherein the algal cells are obtained from a host marine organism.

18. The culture of claim 17, wherein the host is selected from the group consisting of: Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Pocillopora, Rhodactis, Stylophora, Tridacna, and Zoanthus.

19. The culture of claim 17, wherein the host is selected from the group consisting of: Pseudopterogorgia elisabethae, Eunicea fusca, and Pseudopterogorgia bipinnata.

20. The culture of claim 15, wherein the Symbiodinium is of clade B.

21. A method of isolating a biologically active compound, the method comprising the steps of:
providing a marine organism comprising algal cells comprising the biologically active compound;
separating the algal cells from the remainder of the marine organism; and
isolating the biologically active compound from the marine organism.

22. The method of claim 21, wherein the algal cells are of the genus Symbiodinium.

23. The method of claim 22, wherein the species of Symbiodinium is selected from the group consisting of S. kawagutii, S. goreaui, S. muscatinei, S. pulchrorum, S. bermudense, S. californium, S. microadriatiucum, S. pilosum, S. meandrinae, S. corculorum, and S. linucheae. S. microadricum.

24. The method of claim 21, wherein the algal cells are obtained from a host marine organism.

25. The method of claim 24, wherein the host is selected from the group consisting of: Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Pocillopora, Rhodactis, Stylophora, Tridacna, and Zoanthus.

26. The method of claim 24, wherein the host is selected from the group consisting of: Pseudopterogorgia elisabethae, Eunicea fusca, and Pseudopterogorgia bipinnata.

27. The method of claim 22, wherein the Symbiodinium is of clade B.
